(19) United States
(12) Patent Application Publication
(10) Pub. No.: US 2004/0002680 A1
(43) Pub. Date: Jan. 1, 2004

(54) Single lumen balloon catheter apparatus

(75) Inventors: Bernard Ackerman, Metuchen, NJ (US); Robert M. Landis, Mountainside, NJ (US)

Correspondence Address: LATHROP & GAGE LC, 2345 GRAND AVENUE, SUITE 2800, KANSAS CITY, MO, 64108, US

(73) Assignee: Ackrad Laboratories, Inc.

(21) Appl. No.: 10/186,836

(22) Filed: Jul. 1, 2002

(51) Int. Cl. A61M 29/00

(52) U.S. Cl. 604/96.01

(57) ABSTRACT

A catheter useful for non-surgical entry into the cervical canal of a uterus to dispense a diagnostic fluid therein. The catheter includes a tubular body having a lumen extending from a first end thereof to a second end thereof. The lumen includes a first aperture at or adjacent to the first end for dispensing a diagnostic fluid into the interior of a subject uterus, and a balloon disposed marginally adjacent to the first end of the body for fluid sealing the interior of the subject uterus. The lumen further includes a second aperture in fluid communication with the interior of the balloon for inflation thereof with the diagnostic fluid. The area of the first aperture is less than the area of the second aperture to provide a backpressure that inflates the balloon when a diagnostic fluid is injected through the catheter. In most applications, the catheter can be combined with a syringe to form a catheter apparatus.

### RELATED APPLICATIONS

This application is related to copending U.S. patent application Ser. No. 09/449,096, entitled “Single Lumen Balloon Catheter Apparatus”, filed Nov. 24, 1999, and assigned to Ackrad Laboratories, Inc, the assignee herein.

### FIELD OF THE INVENTION

The present invention relates to catheters, and in particular, to a balloon-bearing single lumen catheter for injecting diagnostic fluids into a body cavity and a catheter apparatus employing same.

### BACKGROUND OF THE INVENTION

Diagnostic procedures which require a non-surgical entry into the uterus are well known. One such procedure known as hysterosalpingography, is a radiographic method for imaging the anatomical structures of the uterus and fallopian tubes. Hysterosalpingography involves inserting a fine flexible catheter through the cervical canal and injecting a contrast medium, such as an iodinated fluid, into the uterus. Radiography is then carried out to provide imaging information pertaining to the subject uterus.

Another well known diagnostic procedure which entails the non-surgical entry into the uterus is called hysterosonography. This procedure also employs a fine flexible catheter that is inserted into the cervical canal of the uterus. The catheter in this procedure enables the physician or technician to inject a sterile saline solution into the uterus to expand it so that an ultrasound scanner can be used to sonographically observe the uterus.

The catheters used in both procedures typically have means for sealing off the uterus before or during injection of the fluid to prevent backflow into the vagina. One such means includes an inflatable intrauterine balloon made from an elastomeric material disposed adjacent the distal tip of the catheter. The catheter includes a first lumen that communicates with the interior of the balloon to enable inflation and deflation with an inflation syringe, and second lumen that is open at the distal tip of the catheter to enable injection of a desired diagnostic fluid into the uterus with a injection syringe.

The balloon catheter is operated by inserting the distal tip thereof through the cervical canal and into the uterus with the intrauterine balloon deflated. The insertion of the distal tip operates to position the deflated intrauterine balloon in the uterus or cervical canal. Once positioned, the inflation syringe is used to inflate the balloon with air or saline to create a seal in the cervical canal and the injection syringe is used to inject the desired diagnostic fluid into the uterus.

One problem associated with balloon catheters of this design is that they are relatively expensive to manufacture because they include two lumens and two separate syringes. Therefore, a less expensive balloon-bearing catheter is desirable.

### SUMMARY OF THE INVENTION

A catheter used for non-surgical entry into a uterus to dispense a diagnostic fluid therein, is disclosed herein, which catheter comprises an elongated body having a single lumen extending continuously from a first end thereof to a second end thereof. The lumen includes a first aperture of a predetermined area associated with the first end for dispensing a diagnostic fluid into the interior of a subject uterus, and a balloon disposed marginally adjacent to the first end of the body for fluid sealing the interior of the subject uterus. The lumen further includes a second aperture of a predetermined area in fluid communication with the interior of the balloon for inflation thereof with the diagnostic fluid. The predetermined area of the second aperture is greater than the predetermined area of the first aperture to provide a communication path for inflating the balloon with the diagnostic fluid by virtue of back pressure in said lumen.

The catheter is typically combined with a syringe to form a catheter apparatus if desired.

### BRIEF DESCRIPTION OF THE DRAWINGS

The advantages, nature, and various additional features of the invention will appear more fully upon consideration of the illustrative embodiments now to be described in detail in connection with accompanying drawings wherein:

FIG. 1 is an elevational view of a catheter apparatus according to an embodiment of the invention;

FIG. 2A is a sectional view through a portion of the catheter showing the balloon in a deflated state;

FIG. 2B is a sectional view through a portion of the catheter showing the balloon in an inflated state;

FIG. 3A is a sectional view showing the catheter with the balloon in the deflated state inserted in the cervical canal of a subject uterus; and

FIG. 3B is a diagrammatic view of the catheter with the balloon during fluid injection in the inflated state, anchoring and sealing the catheter apparatus in the cervical canal of a subject uterus.

It should be understood that the drawings are for purposes of illustrating the concepts of the invention and are not necessarily to scale.

### DETAILED DESCRIPTION OF THE INVENTION

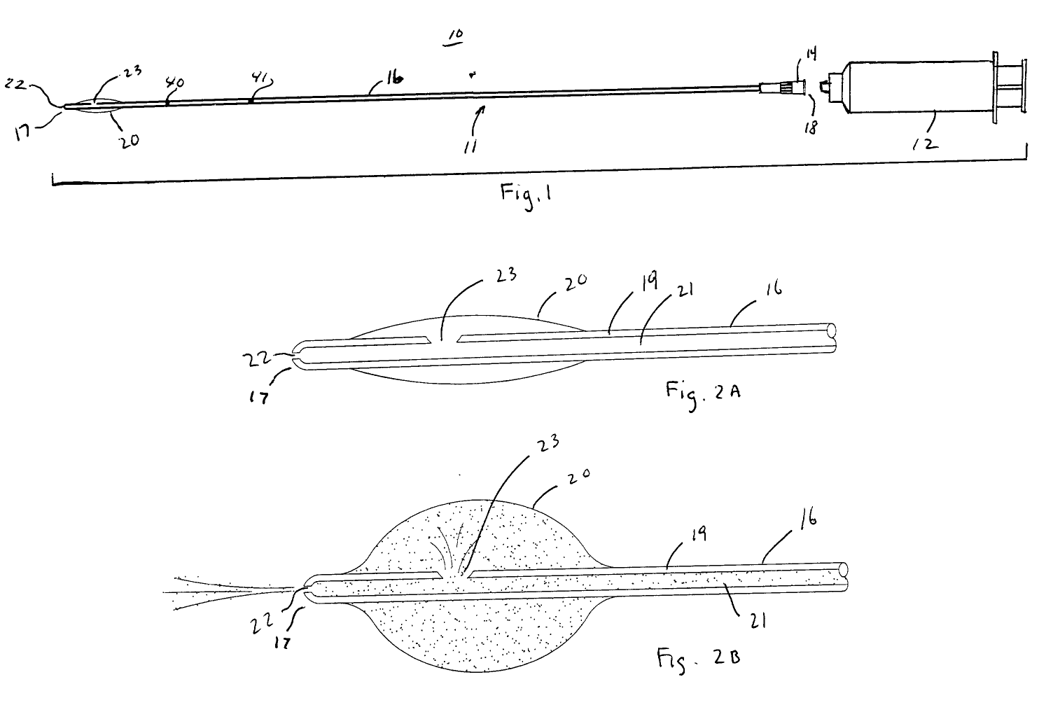

FIG. 1 shows a catheter apparatus according to an embodiment of the invention. The catheter apparatus 10 is an inline assembly comprised of a flexible, single lumen catheter 11 and a conventional syringe 12. The catheter apparatus 10 is primarily intended for non-surgical entry into the cervical canal section of the uterine cavity, however, one of ordinary skill in the art will recognize its usefulness in other related procedures.

The catheter 11 of the apparatus 10 includes an elongated, body 16 with a distal end 17 and a proximal end 18. The catheter body 16 includes first and second marker bands 40 and 41 disposed on its outer surface. The first marker band is about 4 cm from the distal end 17 and the second marker band is about 7 cm from the distal end 17. Each marker band is typically about 5 mm thick and preferably black in color. The body 16 should be made from a material, such as polyurethane, that provides the body 16 with some flexibility, while still allowing the body 16 to be rigid enough to be inserted into the cervical canal without undue bending within the vagina. A conventional female Luer hub connector 14 is typically provided at the proximal end 18 of the catheter body 16 for detachably coupling the syringe 12 (which should be equipped with a male Luer connector) to the catheter 11. An inflatable balloon 20 (shown in the deflated state) is affixed to and encloses a marginal distal end portion of the body 16. The balloon 20 can be of the type described in U.S. Pat. No. 5,624,399 issued to Bernard Ackerman the disclosure of which is incorporated herein by reference.

The balloon 20 of the present invention, as taught in U.S. Pat. No. 5,624,399 is typically constructed from an elastomeric material such as polyurethane or any other elastomeric material having a Shure A durometer of between approximately 70 and 95. U.S. Pat. No. 5,624,399 further teaches attaching the balloon 20 to the body 16 so that its longitudinal axis L is longer than its transverse axis T upon initial inflation thereof. This allows the balloon 20 to be progressively transformed from an ellipsoidal shape to a spherical shape with increasing inflation pressure. The balloon 20 in the ellipsoidal shape as shown in FIGS. 3A and 3B, is used for occluding the cervical canal 32 of a subject uterus 31 thus avoiding visual obstruction of the uterus 31 during imaging. If pain and/or cramping is experienced with the balloon 20 in the cervical canal 32, and this pain and/or cramping can not be substantially mitigated using the method described further on, the balloon can be moved into the entrance of the uterine cavity 33 of the subject uterus 31 and further expanded into the spherical shape to block the internal opening 34 of the cervical canal 32 to obviate the pain and/or cramping.

It should be understood that other embodiments of the invention can employ more conventional balloon designs. Such balloon designs typically inflate into a spherical shape and are made from latex.

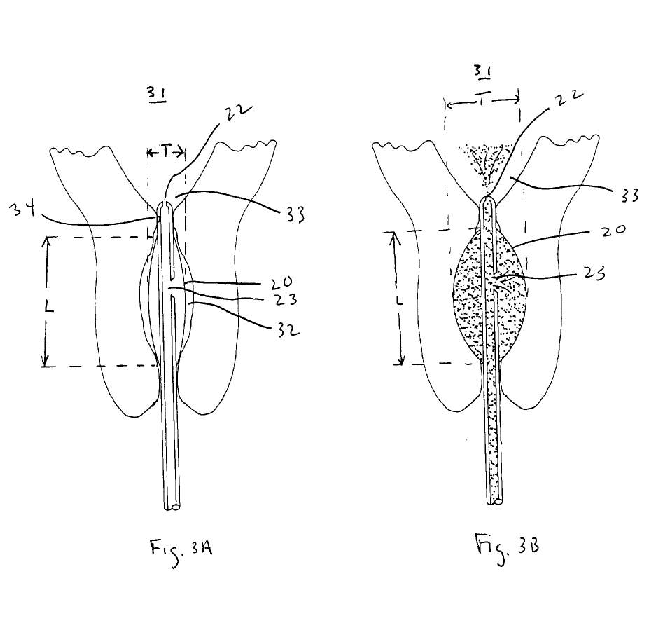

FIG. 2A shows a cross-sectional view through the distal end of the catheter 11 of the apparatus 10. As can be seen, the body 16 of the catheter 11 is constructed with a single lumen 21 that extends continuously the entire length thereof. The wall 19 of the body 16 includes a first aperture 22 at or adjacent to the distal end 17 of the body 16. The first aperture 22 allows the lumen 21 to communicate with the external environment to provide a fluid communication path for injecting a diagnostic fluid such as saline into the uterine cavity of a subject uterus. The lumen 21 also communicates with the interior of balloon 20 via a second aperture 23 provided in the wall 19 of the body 16. The area of the second aperture 23 is larger than the area of the first aperture 22 to provide a communication path for inflating the balloon 20 with diagnostic fluid. Specifically, the first aperture 22 with its smaller area, allows less fluid flow than the larger area second aperture 23. The restricted flow of fluid through the first aperture 22 generates fluid backpressure within the lumen 21 when the syringe 12 is operated. This fluid backpressure causes the diagnostic fluid to flow into the balloon 20 through the second aperture 23, thus, inflating the balloon 20, as shown in FIG. 2B.

In one embodiment, the first and second apertures 22 and 23 are each substantially circular with the first aperture having a diameter that typically ranges between 0.015 and 0.024 inches and produces an area that ranges between 1.77×10⁻⁴ and 4.52×10⁻⁴ square-inches, and the second aperture 23 having a diameter that typically ranges between 0.025 and 0.035 inches and produces an area that ranges between 4.91×10⁻⁴ and 9.62×10⁻⁴ square-inches. It should be understood, however, that one or both apertures 22 and 23 can have other shapes, dimensions and areas that are capable of generating the aforementioned balloon inflating, fluid backpressure.

The apparatus 10 is typically operated by inserting the catheter 11 into the vaginal canal so that the distal end 17 of the catheter 11 enters the cervical canal of a subject uterus. The catheter 11 is then advanced up to the second marker band 41 to safely position the balloon 20 in the uterus. With the balloon 20 positioned in the uterus, the apparatus 10 is stable enough to allow the physician to use both hands to set up the sonographic equipment. The catheter 11 is then partially withdrawn back to the first marker band 40 to position the balloon 20 in the center of the cervical canal, as shown in FIG. 3A.

The syringe 12 of the apparatus 10, which is filled with a diagnostic fluid such as saline, is then operated to inject the diagnostic fluid into the uterine cavity of the uterus. As the syringe is operated, diagnostic fluid flows through the lumen 21 and out the first aperture 22 into the uterine cavity. The fluid backpressure generated within the lumen 21, due to the restricted fluid flow through the first aperture 22, causes the diagnostic fluid to flow into the balloon 20 through the second aperture 23, thereby inflating the balloon 20 as shown in FIG. 3B. Once inflated, the balloon 20 stablizes the position of the apparatus, and prevents leakage of fluid back through the cervical canal, by providing a seal therein. Sonography can then be performed to provide imaging information pertaining to the subject uterus.

The balloon 20 is maintained in the inflated state as long as fluid is being injected into the uterus. The rate of fluid injection controls the fluid pressure generated within the lumen 21, which in turn, controls the inflated size of the balloon 20. If the rate of fluid injection is increased, the fluid pressure within the lumen 21 will be increased, thus increasing the size of the inflated balloon 20. If the rate of fluid injection is decreased, the fluid pressure within the lumen 21 will be decreased, thus, decreasing the size of the inflated balloon 20. Accordingly, if the rate of injection produces an inflated balloon size that causes the patient discomfort, the physician can reduce the rate of injection to reduce the inflated balloon 20 to a size which will reduce patient discomfort, but still occlude the cervical canal. If leakage of fluid is observed, the physician can increase the rate of fluid injection to increase the size of the inflated balloon 20, to re-establish occlusion.

The balloon 20 is deflated by ceasing the injection of fluid. Once the balloon 20 is deflated, the catheter 11 of the apparatus 10 can be fully withdrawn from the cervical canal.

While the foregoing invention has been described with reference to the above embodiments, various modifications and changes can be made without departing from the spirit of the invention. Accordingly, all such modifications and changes are considered to be within the scope of the appended claims.

What is claimed is:

1. A catheter useful for non-surgical entry into a uterus to dispense a diagnostic fluid therein, the catheter comprising:
an elongated body having a lumen extending continuously from a first end thereof to a second end thereof, the body further having a first aperture of a predetermined area opening into the lumen for dispensing a diagnostic fluid into the interior of a subject uterus; and
a balloon disposed marginally adjacent to the first end of the body for fluid sealing the interior of the subject uterus;
the body further having a second aperture of a predetermined area opening into the lumen for inflating the balloon with the diagnostic fluid;
wherein the predetermined area of the second aperture is greater than the predetermined area of the first aperture.

2. The catheter according to claim 1, wherein the body is made from a polyurethane material.

3. The catheter according to claim 1, wherein the balloon can be sequentially inflated into first and second predetermined shapes.

4. The catheter according to claim 3, wherein the first predetermined shape is substantially elliptical and the second predetermined shape is substantially spherical.

5. The catheter according to claim 1, wherein the balloon is made from a polyurethane material.

6. The catheter according to claim 1, wherein the second aperture allows fluid to enter the balloon under pressure to inflate and to maintain inflation of the balloon.

7. A catheter apparatus useful for non-surgical entry into the cervical canal of a uterus to dispense a diagnostic fluid therein, the catheter apparatus comprising:
a catheter comprising:
an elongated body having a lumen extending continuously from a first end thereof to a second end thereof, the body further having a first aperture of a predetermined area opening into the lumen for dispensing a diagnostic fluid into the interior of a subject uterus; and
a balloon disposed marginally adjacent to the first end of the body for fluid sealing the interior of the subject uterus;
the body further having a second aperture of a predetermined area opening into the lumen for inflating the balloon with the diagnostic fluid;
wherein the predetermined area of the second aperture is greater than the predetermined area of the first aperture; and
a syringe for delivering the diagnostic fluid into the catheter.

8. The catheter apparatus according to claim 7, wherein the second aperture allows fluid to enter the balloon under pressure to inflate and to maintain inflation of the balloon.

9. The catheter apparatus according to claim 7, wherein the balloon can be sequentially inflated into first and second predetermined shapes.

10. The catheter apparatus according to claim 9, wherein the first predetermined shape is substantially elliptical and the second predetermined shape is substantially spherical.

11. The catheter apparatus according to claim 7, wherein the balloon is made from polyurethane.

12. The catheter apparatus according to claim 7, wherein the body is made from a polyurethane material.

13. The catheter apparatus according to claim 7, wherein the syringe is removably coupled to the catheter.

14. A catheter useful for non-surgical entry into the cervical canal of a uterus to dispense a diagnostic fluid therein, the catheter comprising:
an elongated body having a lumen extending continuously from a first end thereof to a second end thereof, the body further having an aperture of a first predetermined area opening into the lumen for dispensing a diagnostic fluid into the interior of a subject uterus; and
a balloon disposed marginally adjacent to the first end of the body for fluid sealing the interior of the subject uterus;
the body further having a single aperture of a second predetermined area opening into the lumen for inflating the balloon with the diagnostic fluid;
wherein the aperture of the second predetermined area allows fluid to enter the balloon under pressure to inflate and to maintain inflation of the balloon.

15. The catheter according to claim 14, wherein the body is made from a polyurethane material.

16. The catheter according to claim 14, wherein the balloon can be sequentially inflated into first and second predetermined shapes.

17. The catheter according to claim 16, wherein the first predetermined shape is substantially elliptical and the second predetermined shape is substantially spherical.

18. The catheter according to claim 14, wherein the balloon is made from a polyurethane material.

19. The catheter according to claim 14, further comprising a syringe for delivering the diagnostic fluid into the catheter.

20. The catheter according to claim 19, wherein the syringe is removably coupled to the catheter.

21. A method of non-surgical entry into a uterus of a patient to dispense a diagnostic fluid therein, the method comprising the steps of:
providing a catheter apparatus which comprises a catheter, and a syringe removably coupled to the catheter, the syringe filled with a diagnostic fluid, the catheter including an elongated body having a lumen extending continuously from a first end thereof to a second end thereof, the body having a first aperture of a predetermined area opening into the lumen, the catheter further including a balloon disposed marginally adjacent to the first end of the body, the body further including a second aperture providing fluid communication between the lumen and an interior of the balloon, the second aperture having a predetermined area which is greater than the predetermined area of the first aperture;
positioning the balloon of the catheter in a deflated state in a cervical canal of the uterus;
operating the syringe at a selected injection rate to cause the diagnostic fluid to flow through the lumen of the catheter and out the first aperture into the uterus, the predetermined area of the first aperture generating a fluid pressure within the lumen which is proportional to the selected injection rate, the fluid pressure within the lumen causing the diagnostic fluid to flow into the balloon through the second aperture, thereby inflating the balloon to a size that is proportional to the fluid pressure, the inflated balloon occluding the cervical canal and sealing the uterus.

22. The method according to claim 21, wherein when the patient feels discomfort further comprising the step of reducing the selected injection rate to reduce the fluid pressure within the lumen, thereby reducing the size of the inflated balloon.

23. The method according to claim 22, wherein when fluid leakage is observed through the cervical canal further comprising the step of increasing the selected injection rate to increase the fluid pressure within the lumen, thereby increasing the size of the inflated balloon.

24. The method according to claim 21, wherein when fluid leakage is observed through the cervical canal further comprising the step of increasing the selected injection rate to increase the fluid pressure within the lumen, thereby increasing the size of the inflated balloon.
